(19) United States
(12) Patent Application Publication
(10) Pub. No.: US 2018/0361107 A1
(43) Pub. Date: Dec. 20, 2018

(54) Portable Oxygen System

(71) Applicant: Merle Darling, Antelop, CA (US)

(72) Inventor: Merle Darling, Antelop, CA (US)

(21) Appl. No.: 15/627,991

(22) Filed: Jun. 20, 2017

(51) Int. Cl. A61M 16/20; A61M 16/06; A61M 16/08

(52) U.S. Cl. CPC A61M 16/20; A61M 16/0677; A61M 16/00; A61M 16/207; A61M 16/0841

(57) ABSTRACT

A portable oxygen system for enhancing recovery time during a work out routine includes an oxygen tank that has an outlet. The oxygen tank contains gaseous oxygen. A hose unit is coupled to the outlet and the hose unit is in fluid communication with the oxygen tank. Thus, the hose unit selectively delivers the gaseous oxygen to a user. The hose unit is selectively extended and retracted with respect to the outlet.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIE THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to oxygen devices and more particularly pertains to a new oxygen device for enhancing recovery time during a work out routine.

### BRIEF SUMMARY OF THE INVENTION

An embodiment of the disclosure meets the needs presented above by generally comprising an oxygen tank that has an outlet. The oxygen tank contains gaseous oxygen. A hose unit is coupled to the outlet and the hose unit is in fluid communication with the oxygen tank. Thus, the hose unit selectively delivers the gaseous oxygen to a user. The hose unit is selectively extended and retracted with respect to the outlet.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of a portable oxygen system according to an embodiment of the disclosure showing a hose in a retracted position.

FIG. 2 is a top perspective view of an embodiment of the disclosure showing a hose in an extended position.

FIG. 3 is a front cut-away view of an embodiment of the disclosure.

FIG. 4 is a top phantom view of an embodiment of the disclosure.

FIG. 5 is a perspective in-use view of an embodiment of the disclosure.

### DETAILED DESCRIPTION OF THE INVENTION

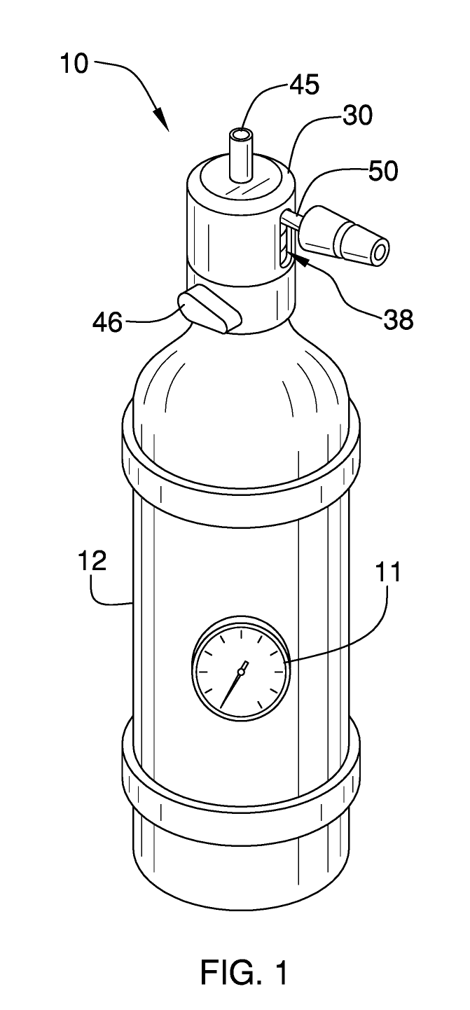

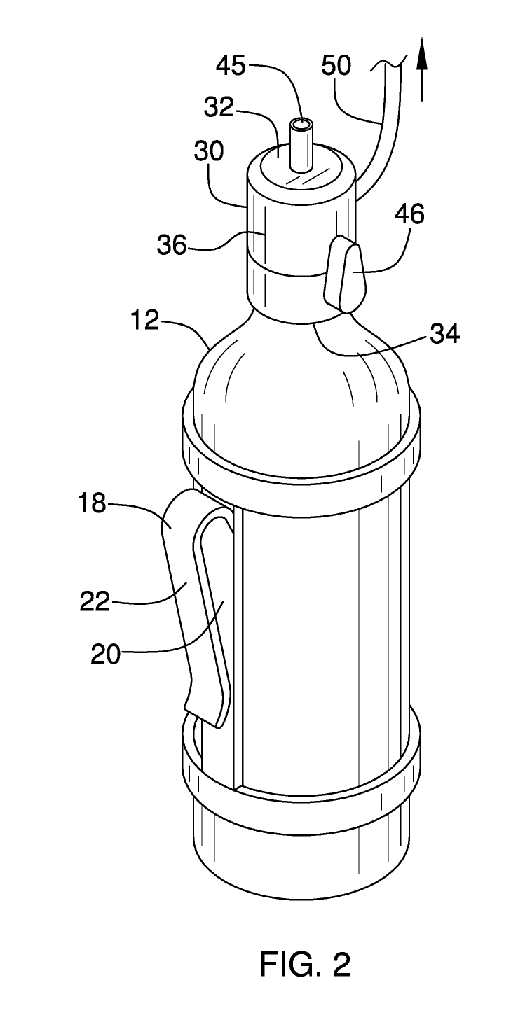

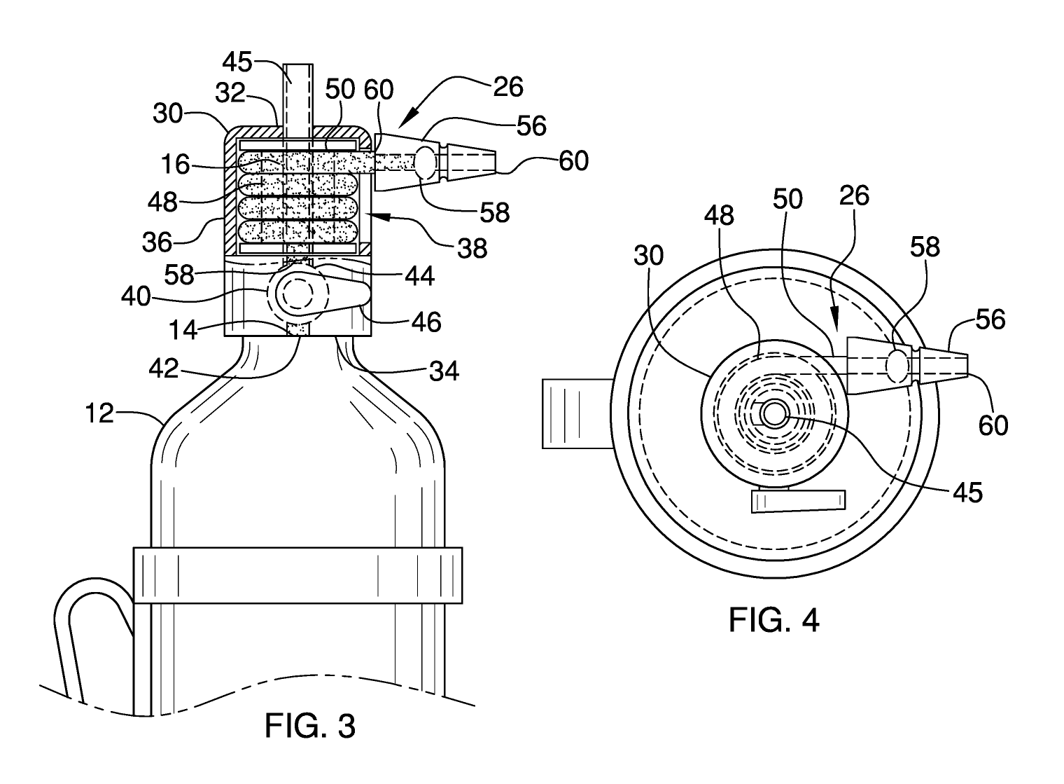

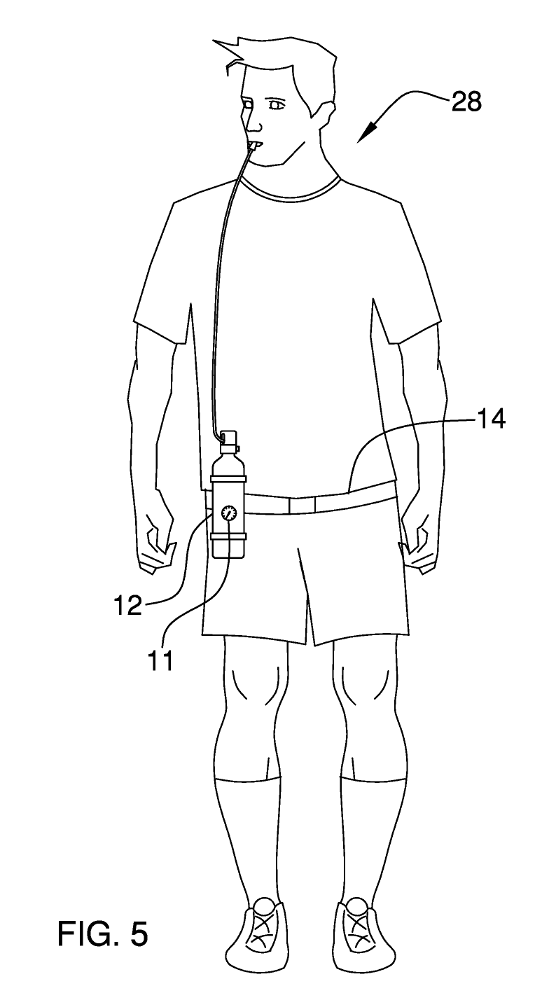

With reference now to the drawings, and in particular to FIGS. 1 through 5 thereof, a new oxygen device embodying the principles and concepts of an embodiment of the disclosure and generally designated by the reference numeral 10 will be described.

As best illustrated in FIGS. 1 through 5, the portable oxygen system 10 generally comprises an oxygen tank 12. The oxygen tank 12 has an outlet 14. Moreover, the oxygen tank 12 contains gaseous oxygen 16. The oxygen tank 12 may have a volume ranging between approximately one liter and two liters. A pressure gauge 11 is coupled to the oxygen tank 12 and the pressure gauge 11 is in fluid communication with an interior of the oxygen tank 12. Thus, the pressure gauge 11 displays how much gaseous oxygen is contained in the oxygen tank 12. The pressure gauge 11 may be an analog pressure gauge, an electronic pressure gauge or any other pressure gauge.

A sleeve 13 is provided and the sleeve 13 has a top end 15. The top end 15 is open and the top end 15 insertably receives the oxygen tank 12 when the oxygen tank 12 is selectively positioned in the sleeve 13. The sleeve 13 is comprised of a thermally insulating material and a deformable material. Thus, the sleeve 13 inhibits heat from heat source from being transferred to the gaseous oxygen 16 in the oxygen tank 12.

A clip 18 is provided. The clip 18 has a first member 20 and a second member 22. The second member 22 is spaced from the first member 20. The first member 20 is coupled to the oxygen tank 12. The second member 22 may engage an article of clothing 24 thereby facilitating the oxygen tank 12 to be retained on the article of clothing 24.

A hose unit 26 is provided. The hose unit 26 is coupled to the outlet 14 such that the hose unit 26 is in fluid communication with the oxygen tank 12. Thus, the hose unit 26 selectively delivers the gaseous oxygen 16 to a user 28. The hose unit 26 is selectively extended and retracted with respect to the outlet 14.

The hose unit 26 comprises a housing 30 that has a top wall 32, a bottom wall 34 and a peripheral wall 36 extending therebetween. The peripheral wall 36 has a slot 38 extending into an interior of the housing 30. A first valve 40 is positioned within the housing 30. The first valve 40 has an intake 42 and an exhaust 44. A fill port 45 is coupled to the top wall 32 of the housing 30 and the fill port 45 is in fluid communication with the outlet 14 when the hose unit 26 is coupled to the outlet 14. The fill port 45 is selectively fluidly coupled to an oxygen source to selectively refill the oxygen tank 12. The fill port 45 may be a gaseous oxygen port of any conventional design.

The intake 42 extends through the bottom wall 34 of the housing 30. The intake 42 is fluidly coupled to the outlet 14 on the oxygen tank 12. Thus, the first valve 40 receives the gaseous oxygen 16. The first valve 40 includes a handle 46. The handle 46 is coupled to the peripheral wall 36 and the handle 46 may be manipulated. The handle 46 selectively opens and closes the first valve 40.

A spool 48 is rotatably positioned within the housing 30. The spool 48 is biased to rotate in a first direction. The spool 48 is selectively urged to rotate in a second direction. The spool 48 is vertically oriented in the housing 30. The spool 48 may be a spring loaded spool or the like.

A hose 50 is provided. The hose 50 has a first end 52 and a second end 54. The first end 52 is fluidly coupled to the exhaust 44. The hose 50 is wrapped around the spool 48 and the second end 54 extends through the slot 38 in the housing 30.

The spool 48 is urged to rotate in the second direction when the second end 54 is urged away from the housing 30. Thus, the second end 54 may be positioned in the user's 28 mouth thereby facilitating the gaseous oxygen 16 to be delivered to the user 28. The spool 48 rotates in the first direction when the second end 54 is released. Thus, the hose 50 is wrapped around the spool 48.

A nozzle 56 is provided and the nozzle 56 is fluidly coupled to the second end 54 of the hose 50. Thus, the nozzle 56 receives the gaseous oxygen 16. A second valve 58 is provided and the second valve 58 is positioned within the nozzle 56. The nozzle 56 has a distal end 60 with respect to the second end 54 and the distal end 60 is open. The nozzle 56 is compressed between the user's 28 teeth to open the second valve 58. Thus, the second valve 58 selectively delivers the gaseous oxygen 16 to the user 28. The second valve 58 may be a bite valve or the like.

In use, the oxygen tank 12 is clipped to the article of clothing 24. The handle 46 is manipulated to open the first valve 40. The hose 50 is selectively urged outwardly from the housing 30 and the nozzle 56 is positioned in the user's 28 mouth. The user 28 bites on the second valve 58 to deliver the gaseous oxygen 16 to the user 28. The gaseous oxygen 16 speeds up muscle recovery when the user 28 is exercising. The user 28 inhales the gaseous oxygen 16 between sets of strength training or the like. Thus, the gaseous oxygen 16 enhances a work out routine.

With respect to the above description then, it is to be realized that the optimum dimensional relationships for the parts of an embodiment enabled by the disclosure, to include variations in size, materials, shape, form, function and manner of operation, system and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded.

A reference to an element by the indefinite article “a” does not exclude the possibility that more than one of the element is present, unless the context clearly requires that there be only one of the elements.

I claim:

1. A portable oxygen system comprising:
an oxygen tank having an outlet, said oxygen tank being configured to contain gaseous oxygen; and
a hose unit being coupled to said outlet such that said hose unit is in fluid communication with said oxygen tank wherein said hose unit is configured to selectively deliver the gaseous oxygen to a user, said hose unit being selectively extended and retracted with respect to said outlet.

2. The system according to claim 1, further comprising a clip having a first member and a second member being spaced from said first member, said first member being coupled to said oxygen tank, said second member being configured to engage an article of clothing thereby facilitating said oxygen tank to be retained on the article of clothing.

3. The system according to claim 1, wherein said hose unit comprises a housing having a top wall, a bottom wall and a peripheral wall extending therebetween, said peripheral wall having a slot extending into an interior of said housing.

4. The system according to claim 3, further comprising a first valve being positioned within said housing, said first valve having an intake and an exhaust, said intake extending through said bottom wall of said housing, said intake being fluidly coupled to said outlet on said oxygen tank wherein said first valve is configured to receive the gaseous oxygen.

5. The system according to claim 4, wherein said first valve includes a handle being coupled to said peripheral wall wherein said handle is configured to be manipulated, said handle selectively opening and closing said first valve.

6. The system according to claim 3, further comprising a spool being rotatably positioned within said housing, said spool being biased to rotate in a first direction, said spool being selectively urged to rotate in a second direction, said spool being vertically oriented in said housing.

7. The system according to claim 6, further comprising:
a first valve having an exhaust; and
a hose having a first end and a second end, said first end being fluidly coupled to said exhaust, said hose being wrapped around said spool, said second end extending through said slot in said housing, said spool being urged to rotate in said second direction when said second end is urged away from said housing wherein said second end is configured to be positioned in the user's mouth thereby facilitating the gaseous oxygen to be delivered to the user, said spool rotating in said first direction when said second end is released such that said hose is wrapped around said spool.

8. The system according to claim 7, further comprising a second valve being fluidly coupled to said second end of said hose wherein said second valve is configured to receive the gaseous oxygen, said second valve being configured to be manipulated thereby facilitating said second valve to selectively deliver the gaseous oxygen to the user.

9. A portable oxygen system comprising:
an oxygen tank having an outlet, said oxygen tank being configured to contain gaseous oxygen;
a clip having a first member and a second member being spaced from said first member, said first member being coupled to said oxygen tank, said second member being configured to engage an article of clothing thereby facilitating said oxygen tank to be retained on the article of clothing; and
a hose unit being coupled to said outlet such that said hose unit is in fluid communication with said oxygen tank wherein said hose unit is configured to selectively deliver the gaseous oxygen to a user, said hose unit being selectively extended and retracted with respect to said outlet, said hose unit comprising:
a housing having a top wall, a bottom wall and a peripheral wall extending therebetween, said peripheral wall having a slot extending into an interior of said housing,
a first valve being positioned within said housing, said first valve having an intake and an exhaust, said intake extending through said bottom wall of said housing, said intake being fluidly coupled to said outlet on said oxygen tank wherein said first valve is configured to receive the gaseous oxygen, said first valve including a handle being coupled to said peripheral wall wherein said handle is configured to be manipulated, said handle selectively opening and closing said first valve,
a spool being rotatably positioned within said housing, said spool being biased to rotate in a first direction, said spool being selectively urged to rotate in a second direction, said spool being vertically oriented in said housing,
a hose having a first end and a second end, said first end being fluidly coupled to said exhaust, said hose being wrapped around said spool, said second end extending through said slot in said housing, said spool being urged to rotate in said second direction when said second end is urged away from said housing wherein said second end is configured to be positioned in the user's mouth thereby facilitating the gaseous oxygen to be delivered to the user, said spool rotating in said first direction when said second end is released such that said hose is wrapped around said spool, and
a second valve being fluidly coupled to said second end of said hose wherein said second valve is configured to receive the gaseous oxygen, said second valve being configured to be manipulated thereby facilitating said second valve to selectively deliver the gaseous oxygen to the user.
